(19) United States
(12) Patent Application Publication
(10) Pub. No.: US 2025/0221818 A1
(43) Pub. Date: Jul. 10, 2025

(54) INTRAOCULAR LENS INSERTION SYSTEM WITH GUIDE RAILS

(71) Applicant: JelliSee Ophthalmics Inc., McLean, VA (US)

(72) Inventors: Forrest J. Ellis, McLean, VA (US); Nestor Farmiga, Rochester, NY (US)

(21) Appl. No.: 19/011,859

(22) Filed: Jan. 7, 2025

Related U.S. Application Data
(60) Provisional application No. 63/619,394, filed on Jan. 10, 2024.

(51) Int. Cl. A61F 2/16

(52) U.S. Cl. CPC A61F 2/167

(57) ABSTRACT

An insertion system to insert an intraocular lens in a patient's eye is provided. The system includes an introducer device having a tapering front sheath. The front sheath includes a receptacle at a proximal portion thereof that is sized and configured to receive the intraocular lens and inner peripheral guide rails at an intermediate portion thereof. The inner guide rails have an arcuate configuration and include a first member separated by an inner lumen from a second opposing member. The first and second member extend further into the inner lumen at the proximal end compared to the terminal distal end of the inner peripheral rails. The front sheath of the introducer device further includes a distal tip in fluid communication with the distal end of the inner peripheral guide rails.

### TECHNICAL FIELD

The present disclosure relates to an insertion system to insert an intraocular lens into a patient's eye.

### BACKGROUND

Cataract surgery is one of the most common surgical procedure performed each year throughout the world. Approximately 3 million cataract surgeries are performed each year in the United States of America. A cataract is a gradual clouding of the natural lens of the human eye. This clouding results in blurred vision that is correctable with surgical removal of the cataract and, in most cases, placement of a clear artificial intraocular lens (IOL). Modern IOLs include an optic or lens body for focusing light on the retina of the eye. In addition, IOLs include one or more arms (e.g. haptics) that extend outward from the optic body and that facilitate centering the IOL in the desired location within the eye (typically within the lens capsular bag).

Modern cataract removal surgery is performed through a small incision in the periphery of the cornea. Modern IOLs are designed to be deformed such as rolled or folded to a relatively small profile, and then allowed to return to their original shape within the eye. This reduces trauma and facilitates post-operative healing.

Various inserters are available to both fold an IOL and allow insertion of the IOL into the eye in a controlled manner. Inserters for delivering IOLs into the eye typically employ a handpiece and a cartridge having a hollow insertion tube or cannula through which the folded IOL is passed using a pusher. The cartridges are often fabricated from disposable materials, such as plastics, and remain in a sterile package until ready for coupling with the handpiece. Conventional IOL cartridges include a loading chamber connected to the insertion tube. In many popular versions, the loading chamber is formed by two hinged halves which receive the IOL and which close to fold the IOL. A non-folding cartridge can also be used in which instruments (e.g. forceps) are used to insert the IOL into a proximal or rear opening of the cartridge. The injection tube includes a small diameter distal end that is insertable into the incision within the eye. After mating the cartridge with the handpiece (if a separate cartridge is used), the pusher advances the IOL through the loading chamber and through the insertion tube into the eye. The distal end of the cartridge is beveled into a sharp point that enables insertion through the corneal incision and facilitates controlled expulsion and manipulation of the IOL into the capsular bag. However, sometimes the IOL is damaged during the process of forcing the IOL through the insertion tube. Sometimes, the IOL rolls or folds improperly such that it does not unfold (unroll) in a controlled and desired manner during placement of the IOL into the eye (into the lens capsular bag). Some inserters do without the cartridge and may be reusable.

Current inserters do not have internal elements that simultaneously roll or fold the optic and haptics as the IOL passes along the insertion tube. As such, an inserter that properly and reliably rolls or folds the IOL optic and the haptics is needed to facilitate reliable placement of the IOL in the capsular bag of the eye.

### SUMMARY

An insertion system to insert an intraocular lens in a patient's eye is provided. The insertion device can comprise a pusher and an introducer device. The introducer device can have a back sheath having a longitudinally extending lumen sized and configured to slideably receive the pusher. The introducer device can further include a tapering front sheath having a proximal portion, an intermediate portion, and a distal portion. A receptacle can be located at the proximal portion sized and configured to receive the IOL. Inner peripheral guide rails can be located at the intermediate portion of the front sheath in fluid communication with the receptacle. The inner peripheral guide rails can begin at a proximal end and terminate at a distal end. The inner peripheral guide rails can have an arcuate configuration and can comprise a first member separated by an inner lumen from a second opposing member. The first and second member can extend further into the inner lumen at the proximal end compared to the distal end of the inner peripheral rails. The introducer device can further include a distal tip in fluid communication with the distal end of the inner peripheral guide rails.

### BRIEF DESCRIPTION OF THE DRAWINGS

FIG. 1 is a perspective view of a kit according to the disclosure including the front sheath of an introducer device of an insertion system and an example of an IOL according to an aspect of the present disclosure.

FIG. 2 is a perspective view of the IOL of FIG. 1 loaded into a receptacle of the front sheath of the introducer device of FIG. 1.

FIG. 3 is a side perspective view of an insertion system with an IOL loaded into the receptacle of the front sheath of the introducer device of FIG. 2 according to an aspect of the present disclosure.

FIG. 4A is a partial perspective view of an insertion system depicting a pusher urging an IOL distally in the front sheath depicted in FIG. 3 according to an aspect of the present disclosure.

FIG. 4B is a partial perspective view of an insertion system depicting a pusher urging an IOL further distally in the front sheath depicted in FIG. 4A according to an aspect of the present disclosure.

FIG. 5 is a partial perspective view of an insertion system depicting the front sheath fully snapped into the back sheath of the introducer device of FIG. 4B according to an aspect of the present disclosure.

FIG. 6 is a partial perspective view of the insertion system of FIG. 5 depicting the pusher urging the IOL out of the distal tip of the front sheath of the introducer device according to an aspect of the present disclosure.

FIG. 7 is a partial perspective view of the insertion system of FIG. 6 with the IOL fully ejected from the introducer device according to an aspect of the present disclosure.

FIG. 8 is a perspective view of a front sheath of an introducer device of an insertion system illustrating internal peripheral guide rails according to an aspect of the present disclosure.

FIG. 9 is a top view of the front sheath of the introducer device of the insertion system of FIG. 8.

FIG. 10 is a top view illustrating the exterior of the front sheath of the introducer device of the insertion system of FIG. 9.

FIG. 11 is a cross-sectional view of the front sheath of FIG. 10 along lines A-A.

FIG. 12 is a cross-sectional view of the front sheath of FIG. 10 along lines B-B.

FIG. 13 is a cross-sectional view of the front sheath of FIG. 10 along lines C-C.

FIG. 14 is a cross-sectional view of the front sheath of FIG. 10 along lines D-D.

FIG. 15 is a cross-sectional view of the front sheath of FIG. 10 along lines E-E.

FIG. 16 is a cross-sectional view of the front sheath of FIG. 10 along lines F-F.

FIG. 17 is a cross-sectional view of the front sheath of FIG. 10 along lines G-G.

FIG. 18 is a cross-sectional view of the front sheath of FIG. 10 along lines A-A with an IOL positioned on the guide rails according to an aspect of the present disclosure.

FIG. 19 is a perspective view of a front sheath of an introducer device of an insertion system illustrating internal peripheral guide rails according to an aspect of the present disclosure.

FIG. 20 is a top view of the front sheath of the introducer device of the insertion system of FIG. 19.

FIG. 21 is a top view illustrating the exterior of the front sheath of the introducer device of the insertion system of FIG. 20.

FIG. 22 is a cross-sectional view of the front sheath of FIG. 21 along lines A-A.

FIG. 23 is a cross-sectional view of the front sheath of FIG. 21 along lines B-B.

FIG. 24 is a cross-sectional view of the front sheath of FIG. 21 along lines C-C.

FIG. 25 is a cross-sectional view of the front sheath of FIG. 21 along lines D-D.

FIG. 26 is a cross-sectional view of the front sheath of FIG. 21 along lines E-E.

FIG. 27 is a cross-sectional view of the front sheath of FIG. 21 along lines F-F.

FIG. 28 is a cross-sectional view of the front sheath of FIG. 21 along lines G-G.

FIG. 29 is a cross-sectional view of the front sheath of FIG. 21 along lines A-A with an IOL positioned on the guide rails according to an aspect of the present disclosure.

### DETAILED DESCRIPTION

The present disclosure relates to an IOL insertion system to insert an IOL, including an accommodative IOL, into a patient's eye. As used herein with respect to a described element, the terms “a,” “an,” and “the” include at least one or more of the described element(s) including combinations thereof unless otherwise indicated. Further, the terms “or” and “and” refer to “and/or” and combinations thereof unless otherwise indicated. By “substantially” is meant that the shape or configuration of the described element need not have the mathematically exact described shape or configuration of the described element but can have a shape or configuration that is recognizable by one skilled in the art as generally or approximately having the described shape or configuration of the described element. The terms “first,” “second,” etc. are used to distinguish one element from another and not used in a quantitative sense unless indicated otherwise. Thus, a “first” element described below could also be termed a “second” element. The terms “distal” and “back” refer to a location, position or direction closer to the operator/clinician when the system is in use and the terms “proximal” and “front” refer to a location, position, or direction closer to the patient when the system is in use. The terms “top” “bottom,” “upper,” “lower,” “underneath,” and “over” refer to the position of elements as depicted in the drawings. As used herein a “patient” includes a mammal such as a human being. All IOL insertion systems and kits as described herein are used for medical purposes and are therefore sterile.

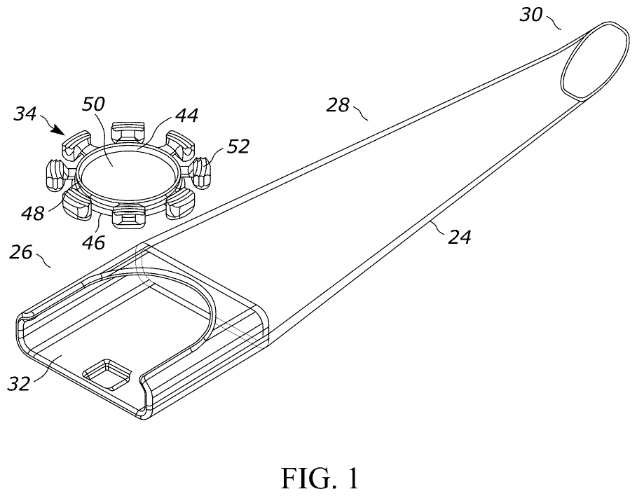

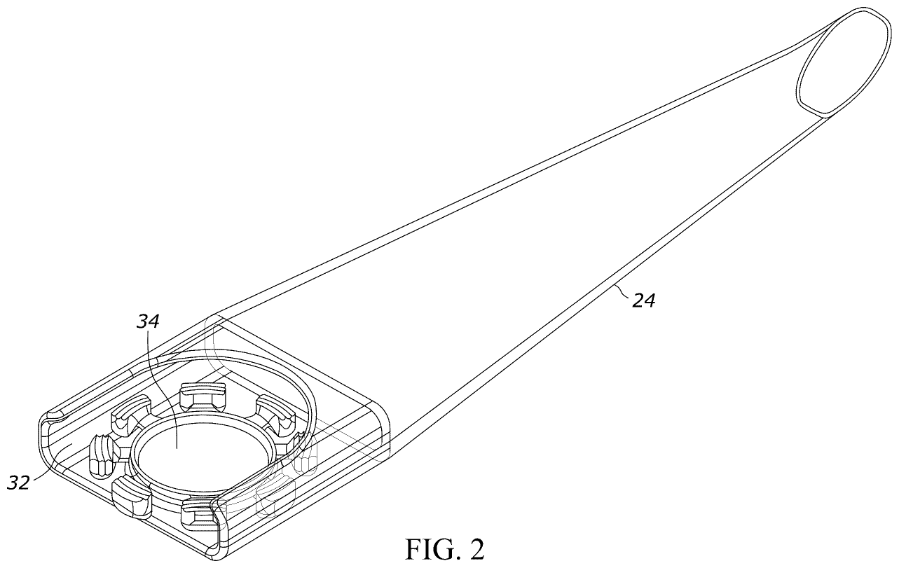

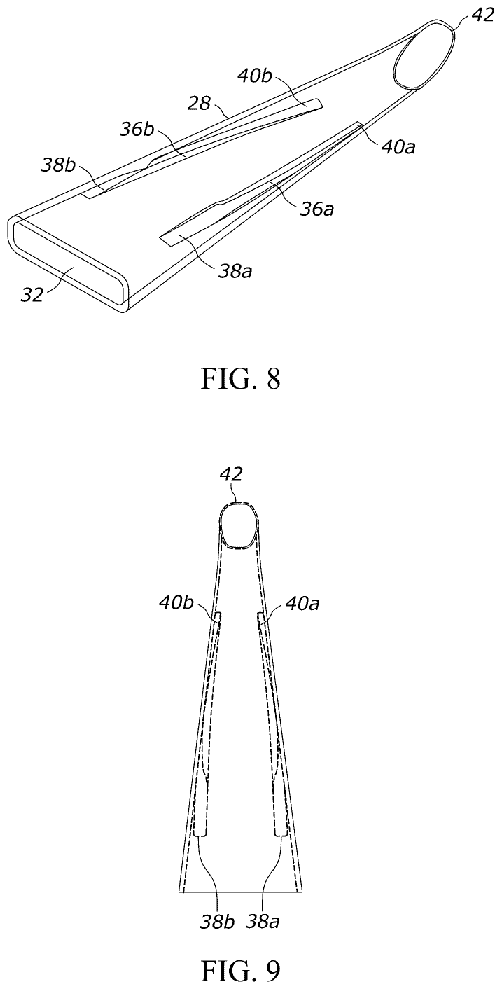

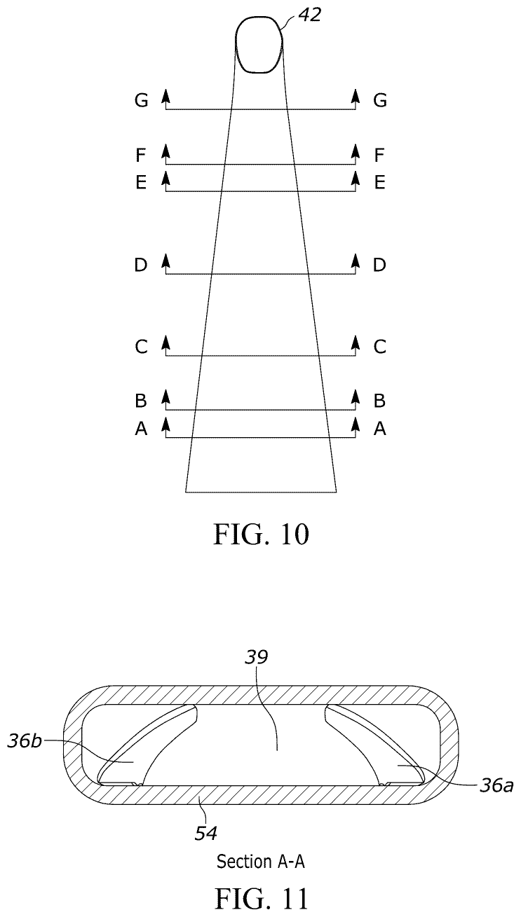

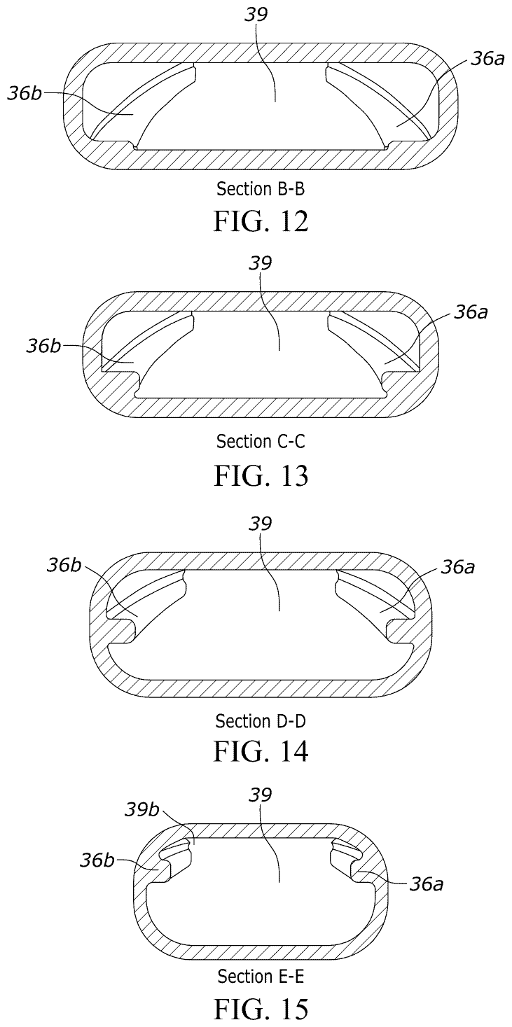

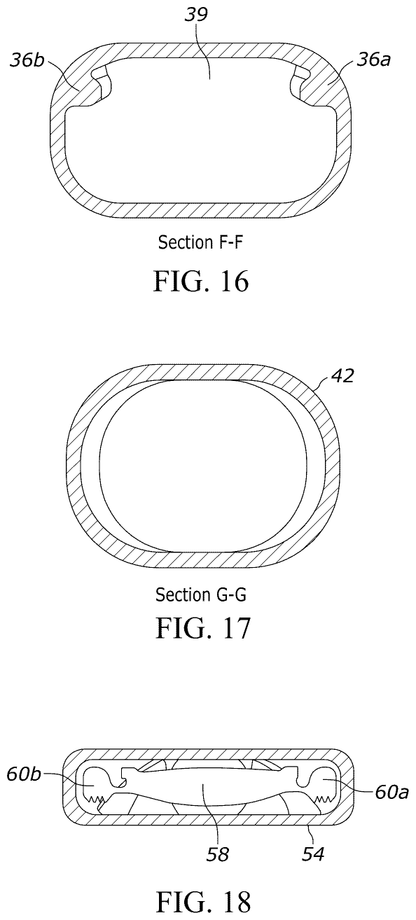

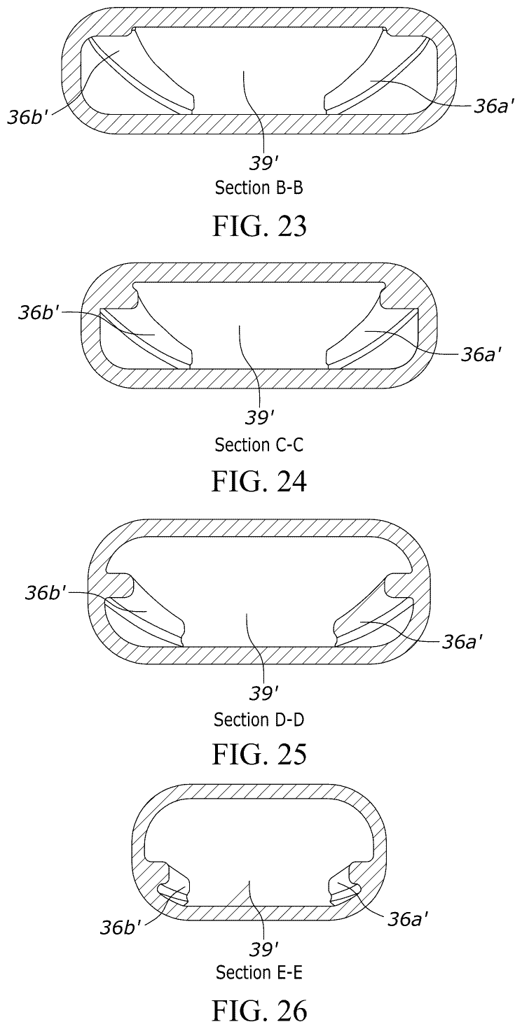

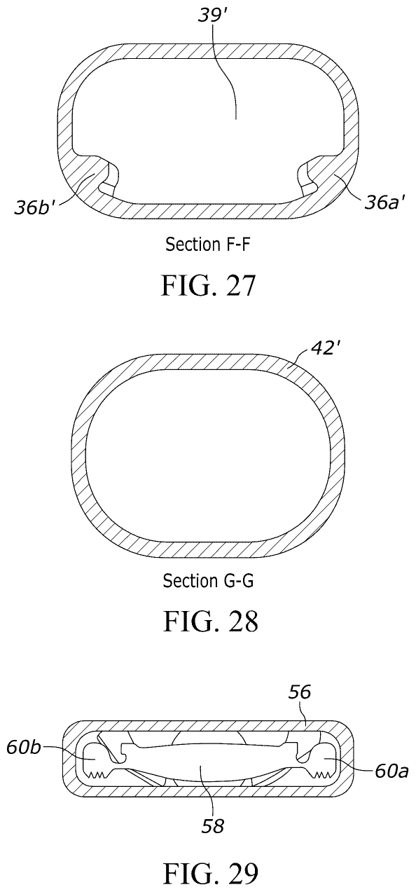

The present disclosure relates to an insertion system for inserting an IOL into a patient's eye. Referring to FIG. 1-8, in an aspect, an insertion system 10 comprises pusher 12 and introducer device 21. Pusher 12 can comprise shaft 14 having actuator 16 at proximal portion 18 thereof. The actuator can be shaped and configured to accommodate a user's finger(s) allowing the user to urge the pusher through the introducer device. Introducer device 21 can comprise back sheath 23 having a longitudinally extending lumen 20 sized and configured to slideably receive pusher 12 and open top portion 22 sized and configured to slideably accommodate the actuator 16. The actuator can have other configurations as well such as, for example, a syringe configuration. Introducer device 21 can further include tapering front sheath 24 having proximal portion 26, intermediate portion 28 and distal portion 30. In certain aspects, the front sheath can taper from a substantially rectangular configuration to a substantially conical or round configuration as shown in FIG. 2. Receptacle 32 can be located at proximal portion 26 sized and configured to receive IOL 34. IOL 34 is only one example of a type of IOL that can be used in conjunction with the insertion system and other configurations of an IOL may also be used. As depicted in FIGS. 8 and 9 and FIGS. 19 and 20, the front sheath can comprise inner peripheral guide rails 36 beginning at proximal end 38 and terminating at distal end 40 and located at intermediate portion 28 of the front sheath in fluid communication with receptacle 32. As shown in FIGS. 11-16 and FIGS. 22-27, inner peripheral guide rails 36 can have an arcuate configuration and can comprise first member 36a and second opposing member 36b. First member 36a can be separated from second member 36b by an inner lumen 39. The first and second member can extend further into the inner lumen at the proximal end compared to the terminal distal end of the inner peripheral rails. The front sheath can further include distal tip 42 in fluid communication with distal end 40 of inner peripheral guide rails 36. In certain aspects, the receptable and the distal tip of the front sheath lack the inner peripheral guide rails. In other words, the inner peripheral guide rails can be confined to the intermediate portion of the front sheath and do not extend into the receptacle and/or the distal tip.

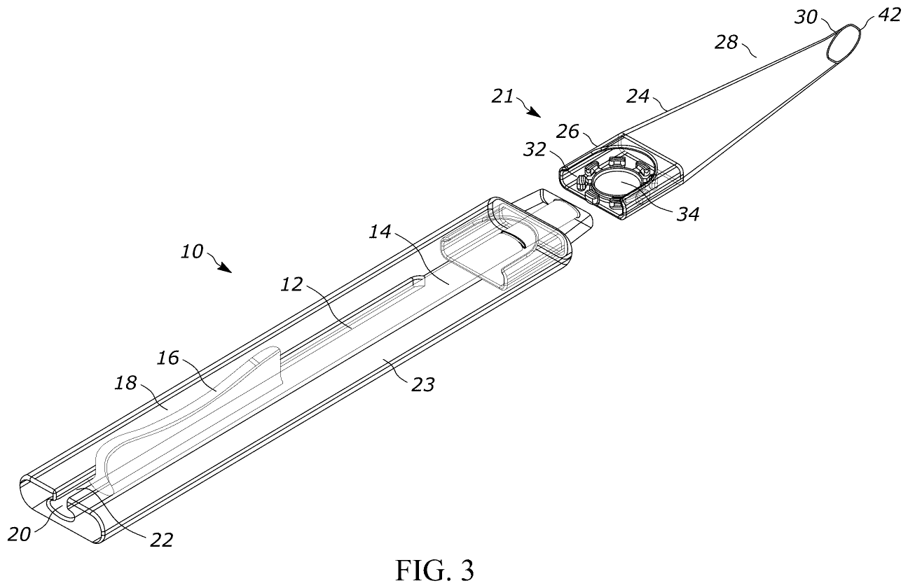

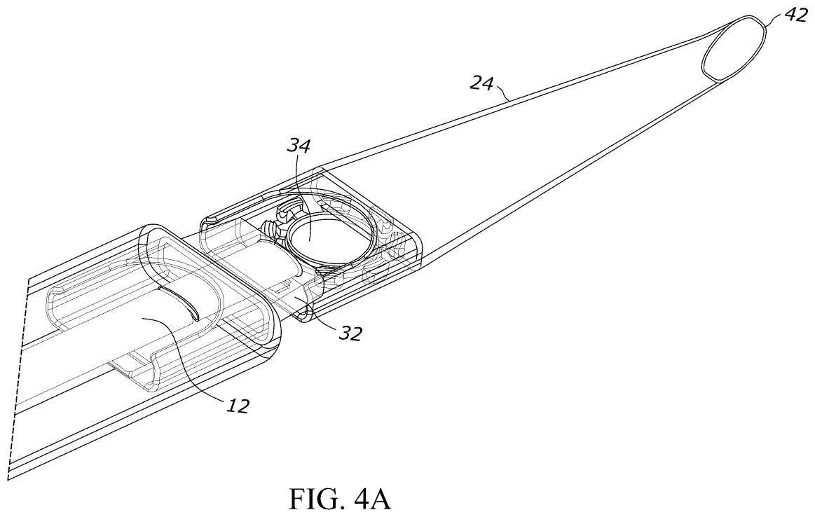

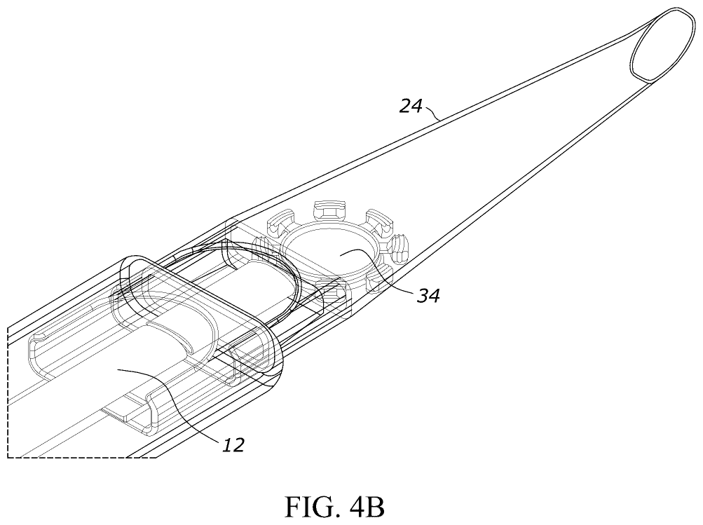

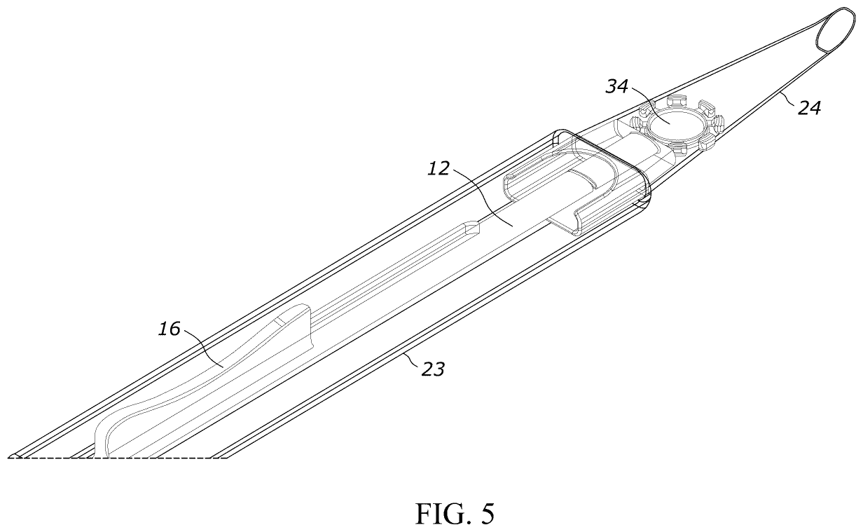

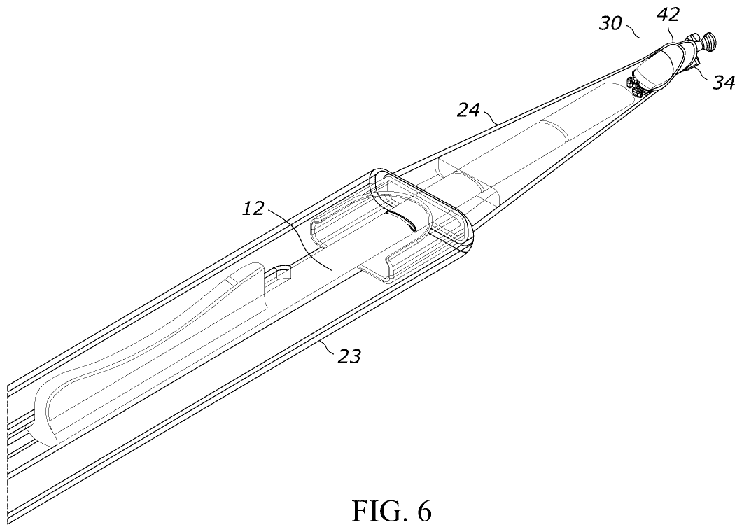

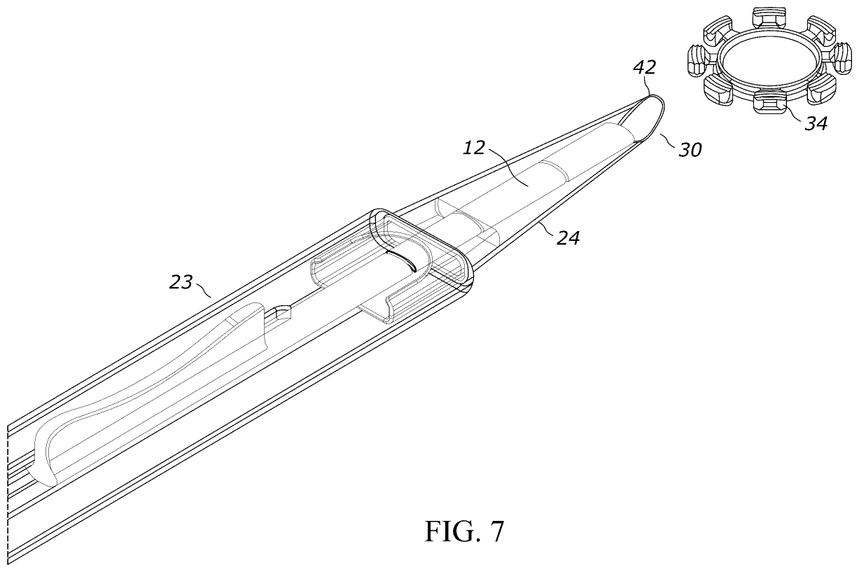

With respect to insertion of an IOL using an example of an insertion system as disclosed herein, FIG. 2 depicts IOL 34 loaded into receptacle 32 of front sheath 24 of introducer device 21. FIG. 3 illustrates insertion system 10 with IOL 34 loaded into receptacle 32 of front sheath 24 of introducer device 21 before pusher 12, which is illustrated as having been positioned in lumen 20 of back sheath 23, has engaged IOL 34. FIG. 4A and 4B depict pusher 12 urging IOL 34 distally in front sheath 24. FIG. 5 illustrates front sheath 24 fully snapped into back sheath 23 of introducer device 21. FIG. 6 depicts pusher 12 urging IOL 34 (in a folded configuration) partially out of distal tip 42 of front sheath 24 of introducer device 21 and FIG. 7 illustrates IOL 34 fully ejected from introducer device 21 and in an unfolded configuration.

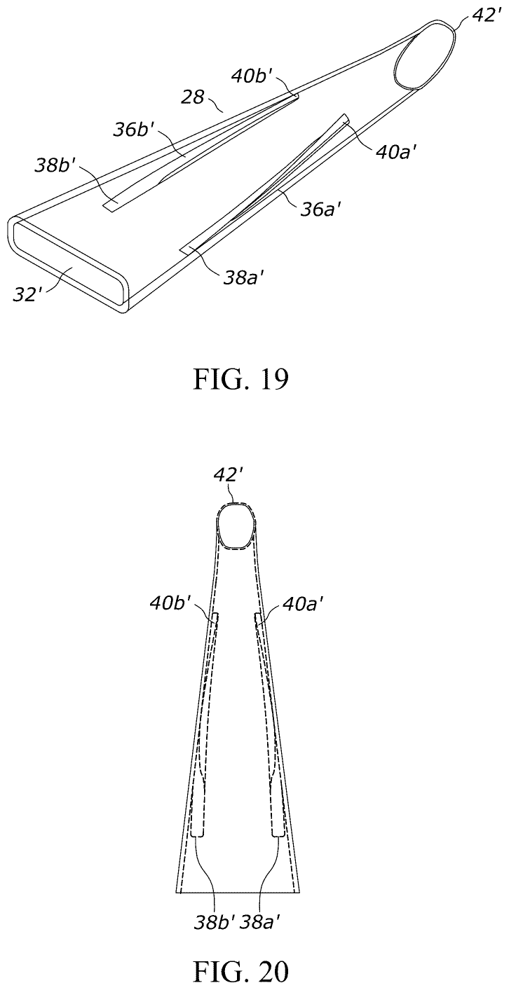

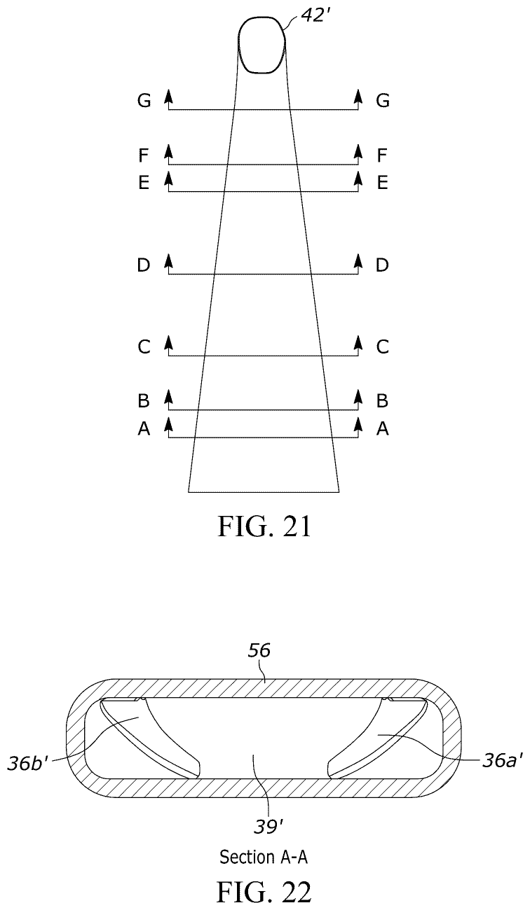

It should be noted that FIGS. 8, 9, 11-16 and 18 illustrate guide rails extending from a lower surface 54 of the intermediate portion of the front sheath whereas FIGS. 19, 20, 22-27 and 29 illustrate guide rails extending from an upper surface 56 of the intermediate portion of the front sheath. As such, with the front sheath of FIG. 8, haptics 60 of IOL 58 fold “over” or on “top” of the guide rails during insertion whereas with the front sheath of FIG. 19, haptics 60 of IOL 58 fold “underneath” or “below” the haptics 60 of IOL 58 during insertion.

In certain aspects, and with reference to FIG. 1, a kit is provided that includes the insertion system disclosed above and an IOL comprising a shape-changing optic configured for placement in or adjacent to a lens capsule, the IOL having an optical axis extending in an anterior-posterior direction, an equator extending in a plane substantially perpendicular to the optical axis, an accommodated state, a dis-accommodated state, and states therebetween. For example, such an IOL can comprise IOL 34, which has an anterior face 44 located anterior to the equator and having a periphery, a posterior face 46 located posterior to the equator and having a periphery, a side wall 48 extending across the equator and extending from the anterior face to the posterior face, a chamber 50 located between the anterior face and the posterior face and containing a material, and at least two opposing haptics 52 connected to the periphery of the anterior face, the posterior face or both. In certain aspects, the at least two opposing haptics comprise a plurality of haptics connected circumferentially about the periphery of the anterior face (as illustrated in FIG. 1), the posterior face, or both. Further details of an example of an IOL are disclosed in U.S. Pat. No. 11,426,272, which is incorporated by reference in its entirety.

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. An insertion system to insert an intraocular lens (IOL) in a patient's eye comprising:
a pusher comprising a shaft;
an introducer device comprising:
a back sheath having a longitudinally extending lumen sized and configured to slideably receive the shaft of the pusher;
a tapering front sheath having a proximal portion, an intermediate portion, and a distal portion and comprising:
a receptacle located at the proximal portion sized and configured to receive the IOL;
inner peripheral guide rails beginning at a proximal end and terminating at a distal end and located at the intermediate portion of the front sheath in fluid communication with the receptacle, the inner peripheral guide rails having an arcuate configuration and comprising a first member separated by an inner lumen from a second opposing member, the first and second member extending further into the inner lumen at the proximal end compared to the distal end of the inner peripheral rails; and
a distal tip lacking in fluid communication with the distal end of the inner peripheral guide rails.

2. The insertion system of claim 1, wherein the front sheath tapers from a substantially rectangular configuration to a substantially conical or rounded configuration.

3. The insertion system of claim 1, wherein the inner peripheral guide rails do not extend into the receptable and the distal tip.

4. A kit comprising:
the insertion system of claim 1;
an IOL comprising:
an anterior face located anterior to the equator and having a periphery;
a posterior face located posterior to the equator and having a periphery;
a side wall extending across the equator and extending from the anterior face to the posterior face;
a chamber located between the anterior face and the posterior face and containing a material; and
at least two opposing haptics connected to the periphery of the anterior face, the posterior face or both.

5. The kit of claim 4, wherein the at least two opposing haptics comprise a plurality of haptics connected circumferentially about the periphery of the anterior face, the posterior face, or both.
